(19) United States
(12) Patent Application Publication
(10) Pub. No.: US 2004/0204712 A1
(43) Pub. Date: Oct. 14, 2004

(54) Bone fixation plates

(76) Inventors: Eric Kolb, Quincy, MA (US); Eric Y. Chan, Quincy, MA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/664,238

(22) Filed: Sep. 17, 2003

Related U.S. Application Data
(63) Continuation-in-part of application No. 10/409,958, filed on Apr. 9, 2003.
(63) Continuation-in-part of application No. 10/609,123, filed on Jun. 27, 2003.

(51) Int. Cl. A61B 17/58

(52) U.S. Cl. 606/69

(57) ABSTRACT

A spinal fixation plate may include a first section having at least one bore formed therein for receiving a bone anchor effective to mate the first section to a first vertebra and a second section having at least one bore formed therein for receiving a bone anchor effective to mate the second section to a second vertebra. At least one of the first section and the second section may have a canted section oriented at a cant angle to at least one other portion of the at least one of the first section and the second section. The cant angle may be selected to correspond to the geometry of at least one of the first vertebra and the second vertebra.

### CROSS REFERENCE TO RELATED APPLICATIONS

The present application is a continuation-in-part of U.S. patent application Ser. No. 10/409,958, entitled Drill Guide and Plate Inserter, filed Apr. 9, 2003, and U.S. patent application Ser. No. 10/609,123, entitled Tissue Retractor and Drill Guide, filed Jun. 27, 2003. Each of the aforementioned patent applications is incorporated herein by reference.

### BACKGROUND

Advancing age, as well as injury, can lead to changes in the bones, discs, joints, and ligaments of the spine, producing pain from nerve compression. Under certain circumstances, alleviation of pain can be provided by performing spinal fusion. Spinal fusion is a procedure that generally involves the removal of the disc between two or more adjacent vertebrae and the subsequent joining of the vertebrae with a bone fixation device to facilitate growth of new osseous tissue between the vertebrae. The new osseous tissue fuses the joined vertebrae such that the vertebrae are no longer able to move relative to each other. Bone fixation devices can stabilize and align the injured bone segments to ensure the proper growth of the new osseous tissue between the damaged segments. Bone fixation devices are also useful for promoting proper healing of injured or damaged vertebral bone segments caused by trauma, tumor growth, or degenerative disc disease.

One such bone fixation device is a bone fixation plate that is used to stabilize, align, and, in some cases, immobilize adjacent skeletal parts such as bones. Typically, the fixation plate is a rigid metal or polymeric plate positioned to span bones or bone segments that require stabilization, alignment, and/or immobilization with respect to one another. The plate may be fastened to the respective bones, usually with bone screws, so that the plate remains in contact with the bones and fixes them in a desired position. Bone plates can be useful in providing the mechanical support necessary to keep vertebral bodies in proper position and bridge a weakened or diseased area such as when a disc, vertebral body or fragment has been removed or during spinal fusion.

Such plates have been used to stabilize, align, and/or immobilize a variety of bones, including vertebral bodies of the spine. For example, a bone plate may include a plurality of screw openings, such as holes or slots, for screw placement. The bone plate may be placed against the damaged vertebral bodies and bone screws or other bone anchors can be used to secure the bone plate to the vertebral bodies. In the case of spinal fusion, for example, a prosthetic implant or bone graft may be positioned between the adjacent vertebrae to promote growth of osseous tissue and fusion of the vertebrae.

One problem with conventional bone plates is that the bone plates often do not conform to the shape of bones, e.g., the vertebral bodies in spinal procedures, to which the plate is attached. As a result, proper placement and fixation of the bone plate to the bone can be difficult.

In spinal fusion procedures, conventional bone plates generally immobilize the connected vertebral bodies, imposing a rigid compressive load on the vertebral bodies. Gaps that often develop in the new osseous tissue growing between the vertebrae can result in decoupling of the compressive load on the osseous tissue and the implant or graft positioned between the vertebrae, as conventional rigid bone plates hold the vertebral body at a fixed distance. To address this problem, dynamic plates have been proposed that aim to permit the vertebral bodies to collapse axially during fusion. However, such dynamic plates suffer from many drawbacks, including creating undesirable off-axis instability and causing damage to adjacent, healthy vertebrae that often results in the need for additional surgical procedures.

### SUMMARY

Disclosed herein are bone fixation plates that facilitate the stabilization, alignment and/or immobilization of bone, in particular, one or more vertebral bodies of the spine. The disclosed bone fixation plates may provide rigid and/or dynamic compressive loads on connected bone portions and are configured to facilitate fixation to the bone portions to be stabilized, aligned, and/or immobilized.

In accordance with one exemplary embodiment, a spinal fixation plate may comprise a first section having at least one bore formed therein for receiving a bone anchor effective to mate the first section to a first vertebra and a second section having at least one bore formed therein for receiving a bone anchor effective to mate the second section to a second vertebra. In the exemplary embodiment, at least one of the first section and the second section may have a canted section oriented at a cant angle to at least one other portion of the at least one of the first section and the second section. The cant angle may be selected to correspond to the geometry of at least one of the first vertebra and the second vertebra.

For example, the first section of an exemplary spinal fixation plate may have a first canted section oriented at a cant angle to the longitudinal axis of the plate and the second section may have a second canted section positioned distal to the first canted section along the longitudinal axis of the plate and oriented at the cant angle to the longitudinal axis of the plate. The cant angle is preferably selected to correspond to the geometry of the first and second vertebrae and thereby facilitate fixation of the plate to the first and second vertebrae.

In another exemplary embodiment, a spinal fixation plate may comprise a first section having at least one bore formed therein for receiving a bone anchor effective to mate the first section to a first vertebra and a second section having at least one bore formed therein for receiving a bone anchor effective to mate the second section to a second vertebra. In the exemplary embodiment, at least one of the second section and the first section may be adjustable with respect to the other section along a longitudinal axis of the plate. A polyaxial bushing is preferably mounted in at least one bore of the spinal fixation plate. The polyaxial bushing may be configured to permit polyaxial rotation of the bushing within the at least one bore.

In a further exemplary embodiment, a spinal fixation plate may comprise a first section having at least one bore formed therein for receiving a bone anchor effective to mate the first section to a first vertebra and a second section having at least one bore formed therein for receiving a bone anchor effective to mate the second section to a second vertebra. In the exemplary embodiment, the at least one bore of the second section may have a second bore axis that intersects the first bore axis of the first bore on a side of the spinal fixation plate distal to the first and second vertebrae.

The at least one bore of the first section may be positioned proximate to an end on the spinal fixation plate and the at least one bore of the second section may be positioned proximate the other end of the spinal fixation plate. In certain exemplary embodiments, at least one of the first bore axis and the second bore axis may be oriented at an angle other than perpendicular to the longitudinal axis of the spinal fixation plate. The angle of the first bore axis and the second bore axis may be, for example, greater than 70° with respect to the longitudinal axis of the spinal fixation plate.

### BRIEF DESCRIPTION OF THE DRAWINGS

These and other features and advantages of the bone fixation plates disclosed herein will be more fully understood by reference to the following detailed description in conjunction with the attached drawings in which like reference numerals refer to like elements through the different views. The drawings illustrate principles of the bone fixation plates disclosed herein and, although not to scale, show relative dimensions.

FIG. 1 is a perspective view of an exemplary embodiment of a single level dynamic bone fixation plate;

FIG. 2 is a side-elevational view in cross-section of the bone fixation plate of FIG. 1 taken along the line A-A in FIG. 1;

FIGS. 3A and 3B are perspective views of the female section of the bone fixation plate of FIG. 1;

FIGS. 4A and 4B are perspective views of the male section of the bone fixation plate of FIG. 1;

FIG. 5 is a partially schematic side elevational view of the bone fixation plate of FIG. 1, which illustrates the cant angles of the canted sections of the bone fixation plate;

FIG. 6 is a schematic illustrating an exemplary single level bone plate coupled to adjacent vertebrae;

FIG. 7 is a perspective view of a pin for connecting the male section and the female section of the bone fixation plate of FIG. 1;

FIG. 8 is a perspective view of an exemplary polyaxial bushing that is operable to connect a bone anchor, such as a bone screw, to a bone fixation plate;

FIG. 9 is a side elevational view of a exemplary bone screw;

FIGS. 10A and 10B are a perspective view and a side elevational view, respectively, of the polyaxial bushing of FIG. 8 coupled to the bone screw of FIG. 9;

FIG. 11 is a perspective view of an exemplary embodiment of a two level dynamic bone fixation plate;

FIG. 12 is a side elevation view of the bone fixation plate of FIG. 11;

FIG. 13 is a side elevation view in cross section of the bone fixation plate of FIG. 11;

FIGS. 14A and 14B are top views of the bone fixation plate of FIG. 11, illustrating the bone fixation plate in a longitudinally expanded configuration (FIG. 14A) and a longitudinally compressed configuration (FIG. 14B);

FIGS. 15A-15C are perspective views of the intermediate section of the bone fixation plate of FIG. 11;

FIG. 16 is a partially schematic side elevational view of the bone fixation plate of FIG. 11, which illustrates the cant angles of the canted sections of the bone fixation plate;

FIG. 17 is a perspective view of an exemplary embodiment of a two level rigid bone fixation plate; and

FIG. 18 is a side elevational view of the bone fixation plate of FIG. 17.

### DETAILED DESCRIPTION OF EXEMPLARY EMBODIMENTS

Certain exemplary embodiments will now be described to provide an overall understanding of the principles of the structure, function, manufacture, and use of the bone fixation plates disclosed herein. One or more examples of these embodiments are illustrated in the accompanying drawings. Those of ordinary skill in the art will understand that the bone fixation plates specifically described herein and illustrated in the accompanying drawings are non-limiting exemplary embodiments and that the scope of the present invention is defined solely be the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

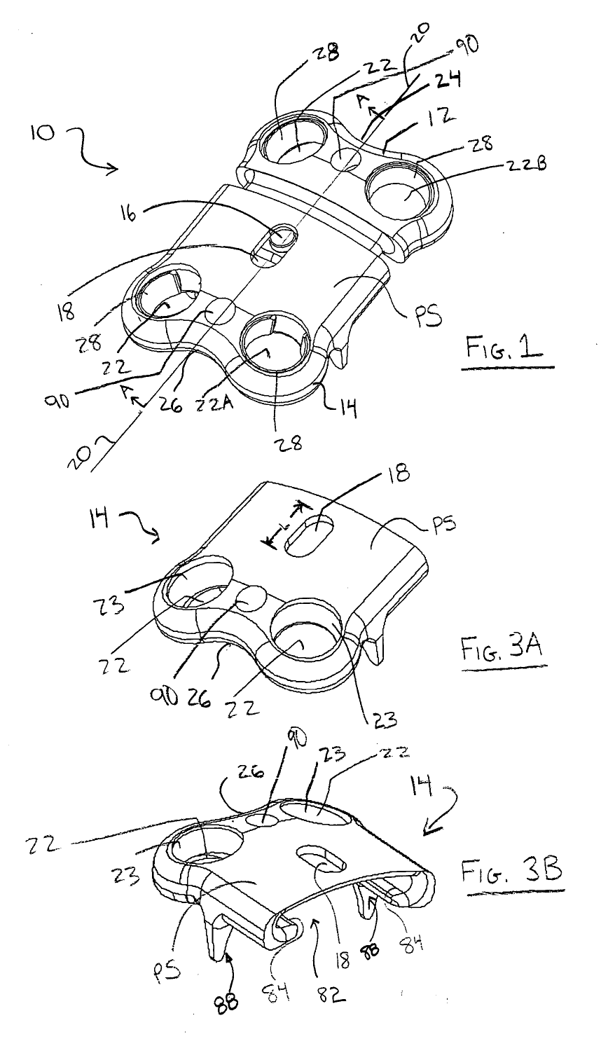

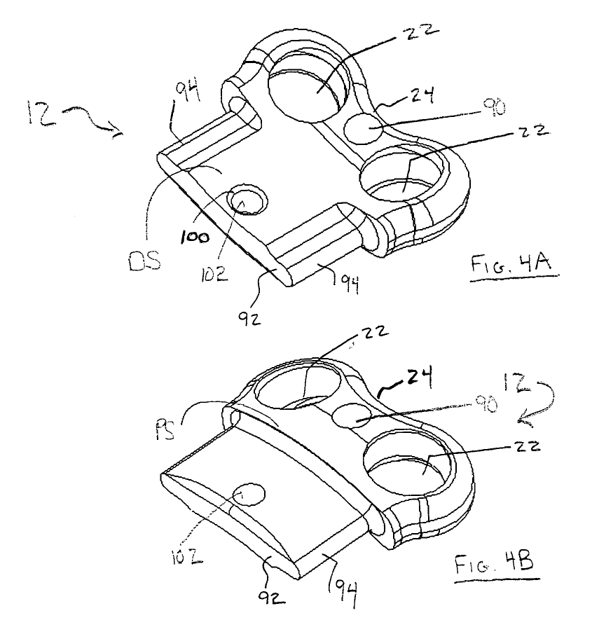

FIGS. 1-5 illustrate an exemplary embodiment of a single level dynamic bone fixation plate 10. The exemplary bone fixation plate 10 is designed to stabilize and align two adjacent bone segments, in particular, two adjacent vertebral bodies (VB₁, VB₂). When implanted, the exemplary bone fixation plate 10 may be fixed at opposing ends to the two adjacent vertebral bodies (VB₁, VB₂) and extend over the disc space (D) between the adjacent vertebral bodies. Although the exemplary bone fixation plate 10 described below is designed primarily for use in spinal applications, such as to stabilize and align adjacent vertebrae to facilitate fusion of the vertebrae, one skilled in the art will appreciate that the structure, features, and principles of the exemplary bone fixation plate 10, as well as the other exemplary embodiments described below, may be applied to any fixation device used to connect two or more sections of bone. Non-limiting examples of applications of the bone fixation plates described herein include long bone fracture fixation/stabilization, small bone stabilization, lumbar spine as well as thoracic stabilization/fusion, cervical spine compression/fixation, and skull fracture/reconstruction plating.

The bone fixation plate 10 has a distal surface (DS) that faces and engages the bone surface upon implantation of the plate and a proximal surface (PS) that faces away from the bone surface and is opposite the distal surface. The term “distal” as used herein with respect to any component or structure will generally refer to a position or orientation that is proximate, relatively, to the bone surface to which bone plate is to be applied. Conversely, the term “proximal” as used herein with respect to any component or structure will generally refer to a position or orientation that is distant, relatively, to the bone surface to which bone plate is to be applied.

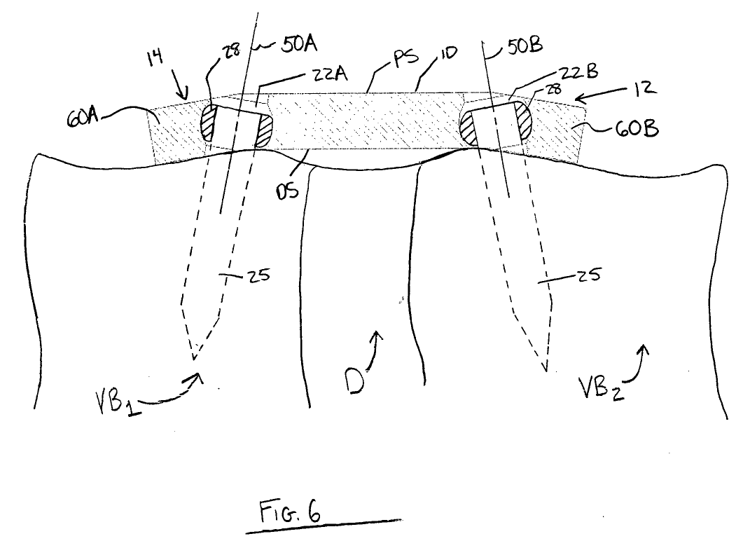

The exemplary bone fixation plate 10 includes two interconnecting sections, a male section 12 and a female section 14, that are dynamically connected through a dynamic connection mechanism, which in the illustrated exemplary embodiment is a rivet-shaped pin 16 (see FIG. 7) that is fixed to the male section 12 and may slide within a longitudinally oriented slot 18 formed within the female section 14. The dynamic connection mechanism allows the male section 12 and the female section 14 to move relative to one another along the longitudinal axis 20 of the bone fixation plate 10.

Continuing to refer to FIGS. 1-4B, the female section 14 receives the male section 12 in a telescoping relationship along the longitudinal axis 20 of the bone fixation plate 10. For example, the female section 14 may have a generally C-shaped cross section that defines a cavity 82 for receiving an interconnect section 92 of the male section 12. In particular, the female section 14 of the exemplary bone fixation plate 10 includes opposing rail guides 84 that are sized to receive rails 94 formed along the opposing sides of the interconnect section 92 of the male section 12. Preferably, the rail guides 84 and the rails 94 are complementary in size and shape to facilitate interconnection therebetween. In the illustrated embodiment, for example, each rail guide 84 has a generally concave, C-shaped cross section and the rails 94 have a generally rounded, concave configuration. The rail guides 84 and the rails 94 are preferably oriented parallel to the longitudinal axis 20 of the bone fixation plate 10, thereby limiting the relative motion of the male section 12 and female section 14 to along the longitudinal axis 20.

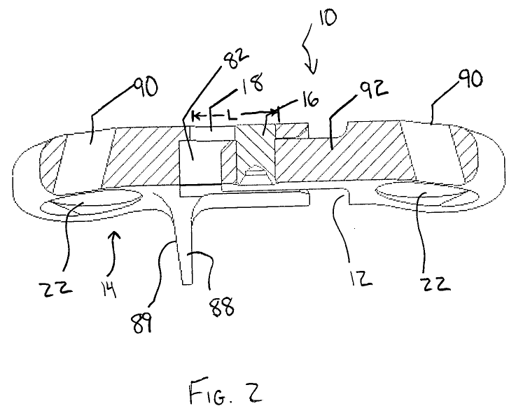

As discussed above, the slot 18 is sized and shaped to receive pin 16 in a sliding relationship. e.g., the pin 16 slides within the slot 18. The length of slot 18, illustrated by arrow L in FIG. 2 and FIG. 3A, as well as the position of the slot 18, may be selected to define the limit of relative motion of the male section 12 and female section 14 along the longitudinal axis 20 of the bone fixation plate 10. For example, selecting a longer slot length may permit greater axial separation of the male section 12 and female section 14.

Referring in particular to FIG. 7, the exemplary pin 16 includes a proximal head 96, a swaged head 98 and a cylindrical shaped shaft 97 extending therebetween. Once swaged during, for example manufacturing of the plate, swaged head 98 fixedly engages the distal end 100 of a hole 102 formed through the interconnect section 92 of the male section 12 to secures the pin 16 to the male section 12. The proximal head 96 of the pin 16 has an outside diameter that is preferably slightly smaller than the width of slot 18. This arrangement allows the proximal head 96 to slide within slot 18. In addition, the slot 18 may be provided with a ratchet mechanism that inhibits movement in one direction along the longitudinal axis. For example, a plurality of ratchet teeth may be formed within slot 18 to engage the pin 16 and inhibit motion of the male section 12 and female section 14 away from one another. Alternatively, a ratchet mechanism may be provided on the guide rails 84 or other interfacing surface of the male section 12 and/or the female section 14.

One skilled in the art will appreciate that other dynamic connection mechanisms may be employed to provide dynamic coupling of the male section 12 and the female section 14. For example, slot 18 may be formed in the male section 12 and pin 16 may be secured to the female section 14. Alternatively, the pin 16 may be provided with external threads for engaging internal threads formed in either the male section 12 or the female section 14.

Moreover, the pin 16, as well as the male section 12 and the female section 14, may be configured to selectively lock the male section 12 and the female section 14 in a desired position with respect to one another. For example, the distal end 98 and/or the proximal end 96 of the pin 16 may be configured to be selectively fixed relative to both sections of the bone fixation plate 10. In one exemplary embodiment, the distal end 98 may be secured to the male section 12 in the manner described above and illustrated in FIGS. 1-4B and 7. In addition, the proximal end 96 of the pin 16 may be configured to be selectively fixed to the female section 14 by, for example, increasing the outer diameter of the proximal end 96 and the length of shaft 97. The bone plate 10 may be converted from a dynamic plate to a rigid plate by advancement of the expanded proximal head 96 into engagement with the female section 14. Prior to advancement, the shaft 97 of the pin 16 may be sized to slide within slot 18 to allow the plate to function as a dynamic plate.

The bone fixation plate 10 may include an alignment mechanism formed on one or both sections 12, 14 of the bone fixation plate 10 to align the bone fixation plate 10 with the end plate of a vertebral body. In the illustrated embodiment, for example, a pair of fins 88 extends from the distal surface of the female section 14 for engagement with the end plate of the vertebral body to which the female section 14 will be connected, as shown in FIGS. 1-3B. Each fin 88 may include a generally planar engagement surface 89 that facilitates engagement with the generally planar anatomy of the end plate of the vertebral body. Optionally, fins 88 may be provided on male section 12 and/or on female section 14. One skilled in the art will appreciate that any number of fins or other alignment mechanisms may be provided to facilitate alignment of the bone fixation plate to bone.

Continuing to refer to FIGS. 1-4B, the exemplary bone fixation plate 10 may include one or more tool holes 90 that facilitate connection of a variety of instruments to the bone fixation plate 10. For example, tool holes 90 may be provided to facilitate connection with a drill guide, a plate inserter, a tissue retractor, or any other instrument used to manipulate the bone fixation plate 10 during implantation. Any number of tool holes 90 may be provided depending, for example, on the size of the bone fixation plate and instruments employed. The size and location of the tool hole(s) may be varied depending, for example, on the size of the bone fixation plate and instruments employed. Notches, cut-out, or the like may be formed along the side edges and end of the bone fixation plate 10, alternative to or in combination with the tool holes, to facilitate connection of a variety of instruments to the bone fixation plate 10.

The exemplary bone fixation plate 10 further includes one or more bores 22 for receiving a bone anchor, such as a bone screw 25, which is effective to mate the bone fixation plate 10 to bone. The bone fixation plate 10 may include any number of bores 22 to fix the plate 10 to bone. The number of bores 22 may vary depending on, for example, the size of the plate, the type(s) of bone anchor(s) employed, and the location and anatomy of bone being secured. In the illustrated exemplary embodiment, the male section 12 includes two bores 22 positioned proximate the end 24 of the male section 12 and the female section 14 includes two bores 22 positioned proximate the end 26 of the female section 14. In each section, the bores 22 are symmetrically positioned about the longitudinal axis of the bone fixation plate 10 and proximate to the ends 24, 26 of the sections, although one skilled in the art will appreciate that other locations are possible.

Moreover, the size and shape of each bore 22 may be selected to match the size and shape of the selected bone anchor. For example, a bore 22 may include internal threads for engagement with threads provided on the bone anchor. Alternatively, in the illustrated exemplary embodiment, each bore 22 may have a generally smooth, e.g., non-textured, interior wall surface 23 that is sized and shaped to receive an expandable polyaxial bushing 28, which is best illustrated in FIGS. 1, 8, 9, 10A and 10B. In particular, each bore may be 22 generally spherical in shape for receiving a polyaxial bushing 28 in a press fit that permits the bushing 28 to rotate within the bore 22 along a plurality of axis prior to radial expansion of the bushing 28. The polyaxial bushing 28 allows a surgeon to select the most desirable angle for the placement of the bone anchor, e.g., a bone screw 25, into bone.

Continuing to refer to FIGS. 8-10B, the illustrated exemplary polyaxial bushing 28 is generally annular in cross-section and may include one or more slots 30 or cutouts that allow for radial expansion of the bushing 28. The bushing 28 may have a generally spherically shaped radial outer surface. The radial outer surface may be roughened by, for example, a plurality of circumferential ridges 32, or other surface texturing, that are configured to grippingly engage the interior wall surface 23 of a bore 22. Radial expansion of bushing 28 expands slot 30 and presses the circumferential ridges 32 against interior wall surface 23 for locking engagement between bushing 28 and bone fixation plate 10. Alternatively, the interior wall surface 23, alone or in combination with the outer surface of the bushing 28, may be textured or roughed to facilitate engagement between the bushing 28 and the bone fixation plate 10. Moreover, in embodiments in which the interior wall surface 23 is not smooth, e.g., it is textured or roughened, the outer surface of the bushing 28 may be smooth, e.g., non-textured or roughened.

The radially interior surface 29 of the illustrated polyaxial bushing 28 defines a passage for receiving the bone anchor having an inner diameter that is preferably less than the outer diameter of the engagement portion of the bone anchor. For example, the head 42 of exemplary bone screw 25 preferably has an outer diameter that is greater than the inner diameter of the passage defined by the radially interior surface 29 of the polyaxial bushing 28. The passage may be, for example, cylindrical in shape or may taper from the proximal end of the bushing to the distal end of the bushing. The bone screw 25 may expand and lock the bushing 28 relative to the plate 10 upon engagement of the head 42 with the bushing 28. In the illustrated exemplary embodiment, the radially interior surface 29 of the illustrated polyaxial bushing 28 and the outer surface of the head 42 are smooth, although, one skilled in the art will appreciate that other surfaces are possible. For example, the radially interior surface 29 of the illustrated polyaxial bushing 28 and the outer surface of the head 42 may both be threaded to permit threaded engagement of the bone screw 25 with the bushing 28.

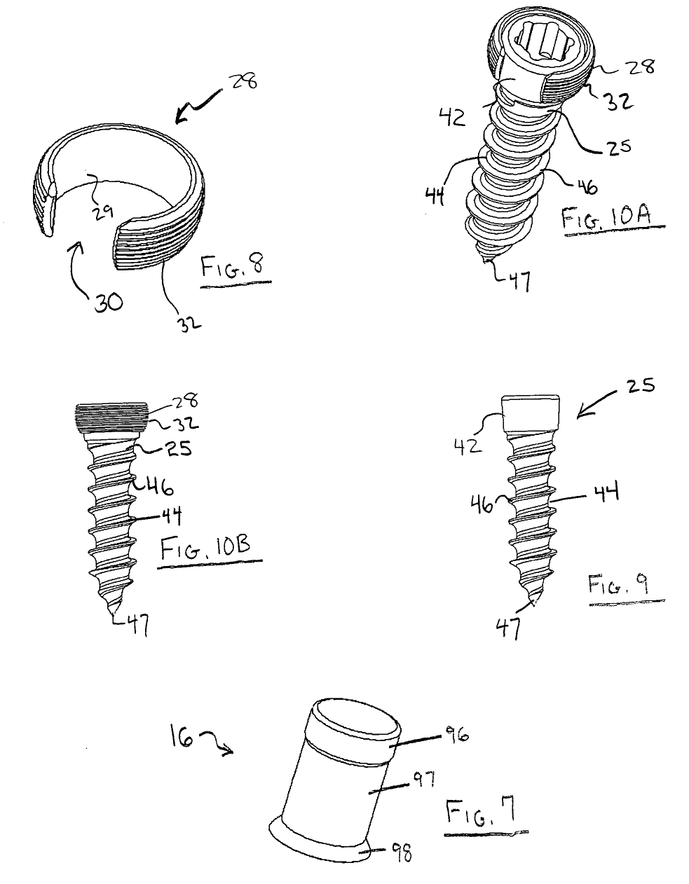

As discussed above, bone screw 25 is formed to engage bushing 28 and to fix the relative positioning of bushing 28 in bore 22. Bone screw 25 is sized for extension through the passageway formed by bushing 28 and for pressing ridges 32 against interior wall surface 23 of bore 22 to form a friction lock between bushing 28 and bone fixation plate 10. As shown in FIGS. 9 and 10A-10B, bone screw 25 includes a threaded distal portion 44 sized for extension through bushing 28 and into bone. The threaded distal portion 44 includes threads 46 extending about an outer surface thereof that terminates at a pointed tip 47 at the distal end of the distal portion 44. Bone screw 25 may be constructed of titanium alloy, although it is understood that bone screw 25 may be constructed of titanium, stainless steel, or any number of a wide variety of materials possessing the mechanical properties suitable for attachment with bone. One skilled in the art will appreciate that other conventional bone anchors may be alternatively employed.

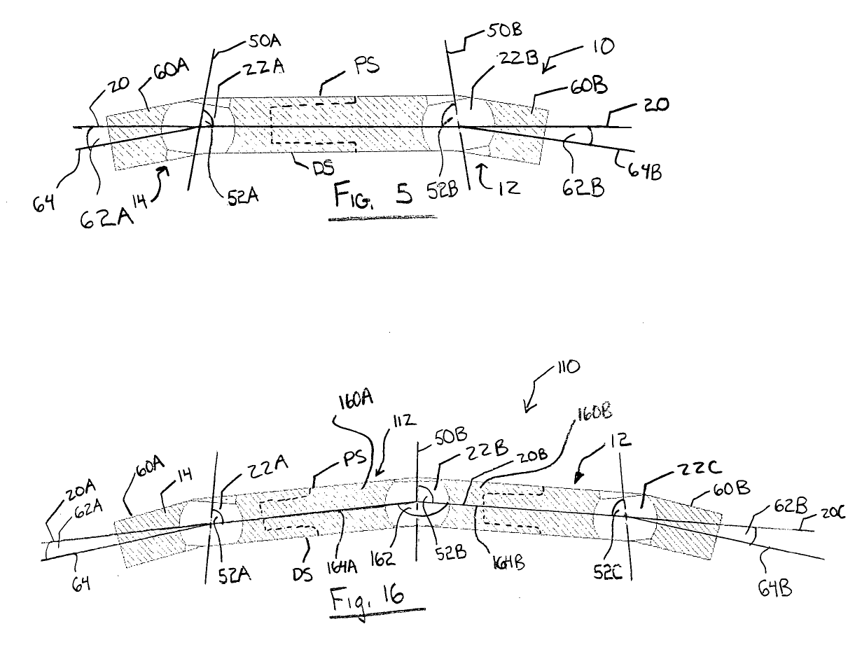

Referring to FIGS. 5 and 6, which provide a partially schematic cross section of the bone fixation plate 10 taken through a line that intersects two bores 22A and 22B at opposing ends of the bone fixation plate 10, each of the bores 22 defines a bore axis 50. The bore axis 50 of one or more of the bores 22 of bone fixation plate 10, or any other dynamic or rigid bone fixation plate disclosed herein, may be varied to provide a range of favored angles for the placement of the bone anchor, e.g., a bone screw 25, into bone. In the exemplary embodiment, bore 22A, which is positioned on the female section 14 of bone fixation plate 10, and bore 22B, which is positioned on the male section 12 of bone fixation plate 10, each define a bore axis 50A, 50B, respectively, that is oriented at a bore angle 52A, 52B, respectively, other than perpendicular to a longitudinal axis 20 of the bone fixation plate 10.

The bore angle 52 can vary depending on, for example, the size of the plate, the bone anchor, and/or the particular application. In certain exemplary embodiments, including the embodiment illustrated in FIG. 5, the bore axis 50A and the bore axis 50B intersect at a point on the proximal side of the bone fixation plate 10. In this configuration, the bone anchors, e.g., bone screws 25, positioned within bores 22A and 22B, i.e., at opposing ends of the bone fixation plate 10, are angled away from one another and away from the center of the bone fixation plate 10. In spinal applications in which the opposing ends of the bone fixation plate 10 are each attached to the vertebral body (VB) of a vertebra as illustrated in FIG. 6, this configuration allows the bone anchors, e.g., bone screws 25, to be angled toward the center of the vertebral body, resulting in better engagement between the bone screws and the vertebral bodies. In the case of cervical plates, for example, the bore angle 52 may be greater that 70° with respect to the longitudinal axis 20 and preferably between 75° and 85°.

The bore axis of each bore provided on a bone fixation plate may have a common bore angle, as in the case of the illustrated exemplary embodiment. Alternatively, the bore angle may vary for each bore provided. Moreover, one skilled in art will appreciate that bore angles other than those illustrated and described herein are possible, including embodiments in which the bore axis 50A and the bore axis 50B intersect at a point on the distal side of the bone fixation plate 10 such that the tips of the bone anchors are angled toward one another. In alternative embodiments, one or more of the bore axes may be oriented parallel to one another. For example, the bore axis 50A and the bore axis 50B may be oriented parallel to one another and at an angle other than perpendicular to the longitudinal axis 20 of the bone fixation plate.

In accordance with the present invention, a bone plate, such as exemplary bone fixation plate 10, may include one or more canted sections 60 that are oriented at a cant angle 62, i.e., an angle other than 0°, to the longitudinal axis of the bone fixation plate 10 or a section of the bone fixation plate 10, as illustrated in FIGS. 5 and 6. The cant angle 62 is preferably selected to correspond to the geometry of the bone to which the bone fixation plate 10 is coupled.

In the illustrated embodiment, for example, the bone fixation plate 10 is provided with a canted section at opposing ends of the bone fixation plate 10. In particular, a canted section 60A is provided at an end of the female section 14 and a canted section 60B is provided at an end of the male section 12. Each canted section 60 defines a cant axis 64 that is oriented at the cant angle 62 with respect to the longitudinal axis 20 of the bone fixation plate 10. Each canted section 60A, 60B may have a common cant angle 62, as illustrated, such that the canted sections are symmetrically oriented with respect to the longitudinal axis 20. Alternatively, one or more canted sections 62 may have distinct cant angles 62.

The cant angle 62 may be selected based on the geometry of the bone to which the bone fixation plate is attached to improve the connection between the bone fixation plate and the bone by increasing the amount of surface contact between the distal surface of the plate and the exterior surface of the bone. In the illustrated exemplary embodiment, for example, each canted section 60 defines a cant axis 64 that is angled distally from longitudinal axis 20 of the bone fixation plate 10. As illustrated in FIG. 6, this configuration of the canted sections 60 corresponds to the geometry of the vertebral body (VB) to which each canted section 60 is coupled. In particular, canted section 60A is angled to correspond to the concave exterior surface of vertebral body VB₁. Likewise, canted section 60B is angled to correspond to the concave exterior surface of vertebral body VB₂. In cervical plates, for example, the cant angle 62 may be less than 20° or in some applications less than 10°. The cant angle 62 for cervical plates is preferably in the range of 3° to 15°, and most preferably is approximately 7°. One skilled in the art will appreciate that cant angles other than those illustrated and described herein are possible. For example, one or more cant sections may define a cant axis that is angled proximally from the longitudinal axis of the bone fixation plate.

Although the cant sections of the illustrated exemplary embodiments are generally linear, one skilled in the art will appreciate that one or more cant sections may be non-linear in configuration. For example, one or more cant sections may be curvilinear.

A cant section may be formed by bending or machining a section of the bone fixation plate to a desired cant angle. Alternatively, a cant section may be formed using a properly shaped mold or cast and through the molding or casting process by which the bone fixation plate is formed.

Although bone fixation plate 10 is illustrated and described, it is understood that bone plates may be formed in any number of shapes and sizes for varying applications. Bone fixation plate 10 may be constructed of a titanium alloy, although it is understood that bone fixation plate 10 may be constructed of titanium, stainless steel, or any number of a wide variety of materials possessing the mechanical properties suitable for coupling bones together.

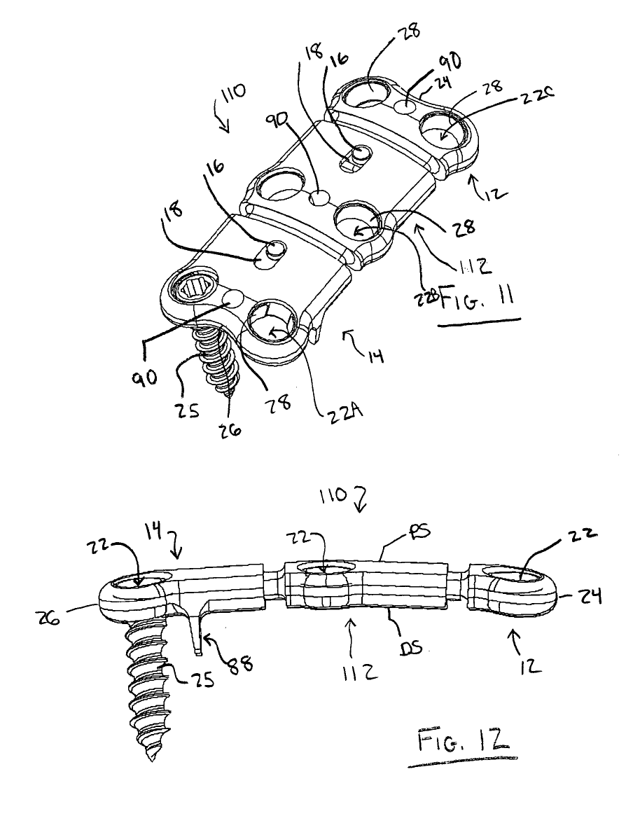

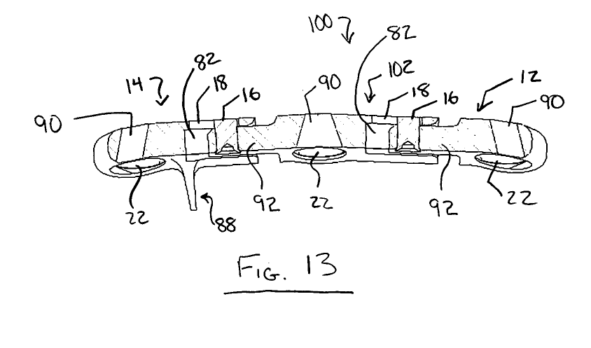

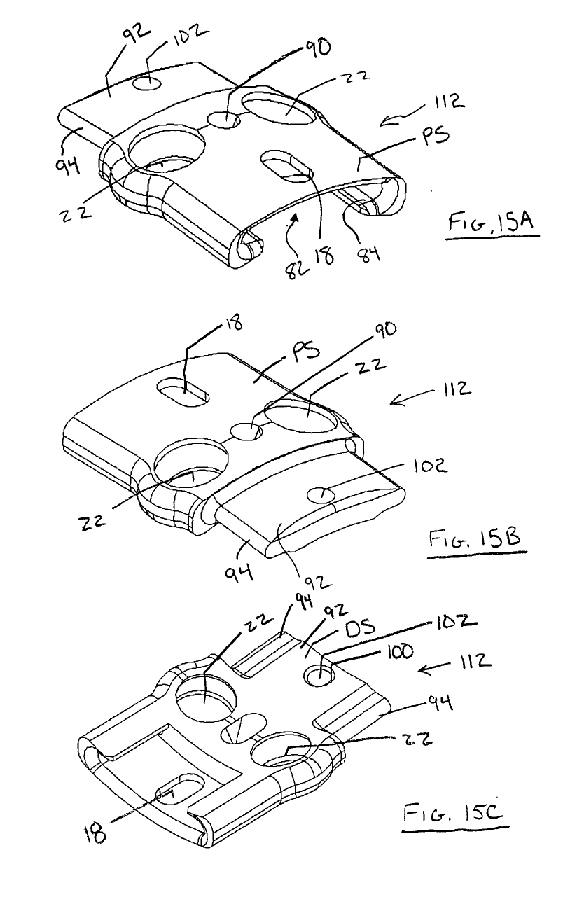

FIGS. 11-17 illustrate an exemplary embodiment of a two level dynamic bone fixation plate 110. The exemplary bone fixation plate 110 is designed to stabilize and align three adjacent bone segments, in particular, three adjacent vertebral bodies (VB₁, VB₂, VB₃). When implanted, the exemplary bone fixation plate 110 may be fixed at opposing ends to two of vertebral bodies (VB₁, VB₃) and in the center at the third vertebral bodies (VB₂) while also extending over the two disc spaces (D₁, D₂) between the three vertebral bodies. The two level bone fixation plate 110 is similar in design and construction to the single level bone fixation plate 10 described above.

The exemplary two level dynamic bone fixation plate 110 includes three interconnecting sections, 110 a male section 12, a female section 14, and an intermediate section 112. The intermediate section 112 is dynamically connected through a pair of dynamic connection mechanisms, for example, pin 16 and slot 18 combination, to both the male section 12 and the female section 14. The dynamic connection mechanisms allow the male section 12 and the female section 14 to move relative to the intermediate member 112, and each other, along the longitudinal axis 20 of the bone fixation plate 110.

Continuing to refer to FIGS. 11-15C, the intermediate section 112 may include components of the male section 12 and the female section 14 to facilitate the dynamic relationship between the three interconnecting sections. In particular, the female section 14 may receive an interconnect section 92 of the intermediate section 112 in a telescoping relationship along the longitudinal axis 20 of the bone fixation plate 10. For example, rails 84 provided on the interconnect section 92 of the intermediate section 112 may be received by guide rails 94 within the cavity 82 of the female section 14. The rail guides 84 and the rails 94 are preferably oriented parallel to the longitudinal axis 20 of the bone fixation plate 110, thereby limiting the relative motion of the female section 14 and intermediate section 112 to along the longitudinal axis.

In addition, the intermediate section 112 may receive the male section 12 in a telescoping relationship along the longitudinal axis 20 of the bone fixation plate 110. For example, the intermediate section 112 may include a cavity 82 having rail guides 84 for receiving rails 94 provided on the interconnect section 92 of the male section 12. The rail guides 84 and the rails 94 are preferably oriented parallel to the longitudinal axis 20 of the bone fixation plate 110, thereby limiting the relative motion of the male section 12 and intermediate section 112 to along the longitudinal axis.

One skilled in the art will appreciate that a multi-level bone fixation plate may be constructed by providing one or more intermediate sections 112. For example, a three level bone fixation plate may be constructed by providing two intermediate sections in addition to a male section 12 and a female section 14.

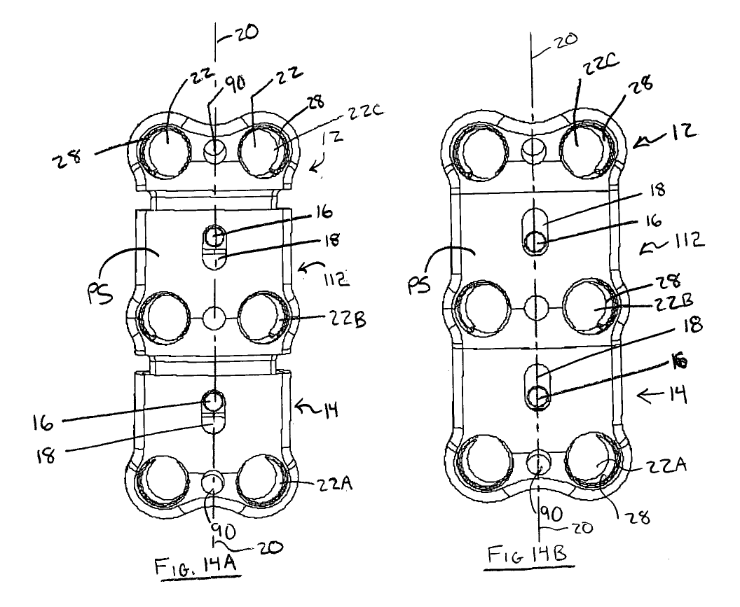

Continuing to refer to FIGS. 11-17, the exemplary two-level bone fixation plate 110 may include one or more bores 22 for receiving a bone anchor, such as a bone screw 25, which is effective to mate the bone fixation plate 110 to bone. In the illustrated exemplary embodiment, for example, the male section 12 includes two bores 22 positioned proximate the end 24 of the male section 12, the female section 14 includes two bores 22 positioned proximate the end 26 of the female section 14, and the intermediate section 112 includes two bores 22 positioned proximate the midpoint of the intermediate member 112. In each section, the bores 22 are symmetrically positioned about the longitudinal axis of the bone fixation plate 110, as best illustrated in FIGS. 14A, 14B, although one skilled in the art will appreciate that other locations are possible.

One or more bores 22 of the exemplary two level bone fixation plate 110 may include a polyaxial bushing 28 to facilitate connection of a bone anchor, e.g., bone screw 25, to the plate 110. Alternatively, one or more bores 22 may be provided with a fixed connection mechanism, e.g., threads, to facilitate connection of a bone anchor, e.g., bone screw 25, to the plate 110.

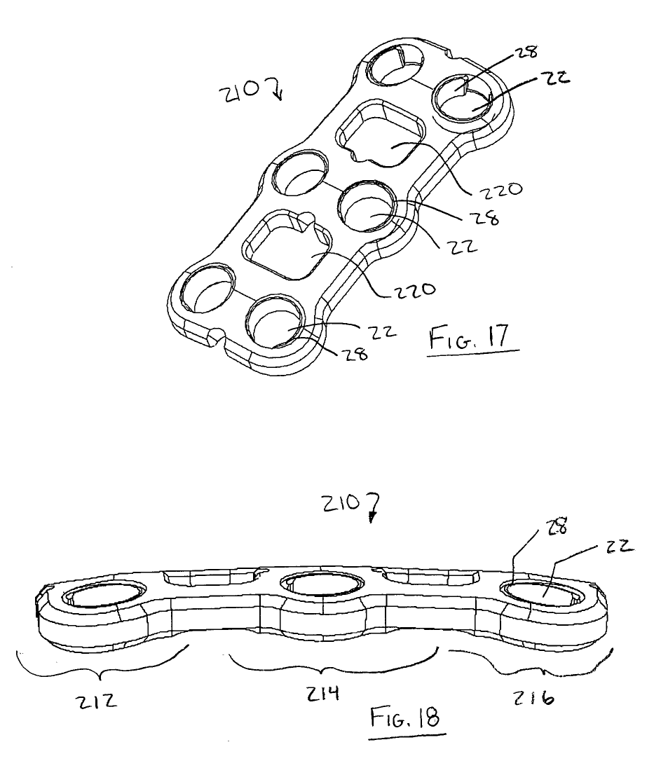

Referring to FIG. 16, which provide a partially schematic cross section of the bone fixation plate 110 taken through a line that intersects three bores 22A, 22B, and 22C of the bone fixation plate 110, each of the bores 22 defines a bore axis 50. As with the exemplary single level bone fixation plate 10, the bore axis 50 of one or more of the bores 22 of bone fixation plate 110 may be varied to provide a range of favored angles for the placement of the bone anchor, e.g., a bone screw 25, into bone. In the exemplary embodiment, bore 22A, which is positioned on the female section 14 of bone fixation plate 110, bore 22B, which is positioned on the intermediate section 112 of bone fixation plate 110, and bore 22C, which is positioned on the male section 12 of bone fixation plate 10, each define a bore axis 50A, 50B, and 50C respectively, that is oriented at a bore angle 52A, 52B, and 52C respectively, other than perpendicular to a longitudinal axis 20A, 20B, 20C of the respective section of the bone fixation plate 110.

The bore angle 52 can vary depending, for example, on the size of the plate, the bone anchor, and/or the particular application. In certain exemplary embodiments, including the embodiment illustrated in FIG. 16, the bore axis 50A and the bore axis 50C intersect at a point on the proximal side of the bone fixation plate 110. In this configuration, the bone anchors, e.g., bone screws 25, positioned within bores 22A and 22C, i.e., at opposing ends of the bone fixation plate 110, are angled away from one another and away from the center of the bone fixation plate 110. In spinal applications in which the opposing ends of the bone fixation plate 110 are each attached to the vertebral body of a vertebra, this configuration allows the bone anchors, e.g., bone screws 25, to be angled toward the center of the vertebral body, resulting in better engagement between the bone screws and the vertebral bodies. In the case of cervical plates, for example, the bore angle 52A and 52C may be greater that 70° and preferably 75° to 85° with respect to the longitudinal axis 20A and 20C of the female section 14 and the male section 12, respectively.

As with the exemplary single level bone fixation plate 10, exemplary two-level bone fixation plate 110 may include one or more canted sections 60 that are oriented at a cant angle 62, i.e., an angle other than 0°, to the longitudinal axis of a respective section of the bone fixation plate 110, as illustrated in FIG. 16. The cant angle 62 is preferably selected to correspond to the geometry of the bone to which the bone fixation plate 110 is coupled.

In the illustrated embodiment, for example, the bone fixation plate 110 is provided with canted sections 60A and 60B at opposing ends of the bone fixation plate 110. In particular, a canted section 60A is provided at an end of the female section 14 and a canted section 60B is provided at an end of the male section 12. Each canted section 60 defines a cant axis 64 that is oriented at the cant angle 62 with respect to the longitudinal axis 20A, 20C of respective section of the bone fixation plate 110.

In the illustrated exemplary two level bone fixation plate 110, for example, canted sections 60A and 60B each define a cant axis 64A, 64B that is angled distally from longitudinal axis 20A, 20C of the respective section (female section 14, male section 12) of the bone fixation plate 110. This configuration of the canted sections 60A and 60B corresponds to the geometry of the vertebral body to which each canted section 60 is coupled. One skilled in the art will appreciate that cant angles other than those illustrated and described herein are possible.

Moreover, the intermediate section comprises two canted sections 160A, 160B that are oriented at a cant angle 162 with respect to each other. Each canted section defines a cant axis 164A and 164B. In the illustrated embodiment, cant axis 164A is coaxial with the longitudinal axis 20A of the female section 14 and cant axis 164B is coaxial with the longitudinal axis 20C of the male section 12. One skilled in the art will appreciate the intermediate section 112 may include a number of canted sections, including one or none, oriented at varying cant angles depending on the geometry of the bone to which the bone fixation plate is attached.

FIGS. 17 and 18 illustrate an exemplary embodiment of a two level rigid bone fixation plate 210. The exemplary bone fixation plate 210 is designed to stabilize and align three adjacent bone segments, in particular, three adjacent vertebrae. When implanted, the exemplary bone fixation plate 210 may be fixed at opposing ends to two of the vertebrae and in the center at the third vertebra while also extending over the two disc spaces between the three vertebrae.

The exemplary two level rigid bone fixation plate 210 includes three interconnecting sections, a first section 212 for connecting to a first vertebra, a second section 214 for connecting to a second vertebra, and a third section 216 for connecting to a third vertebra. In contrast to the dynamic bone fixation plates described above, each section of the exemplary two level rigid bone fixation plate 210 is fixed with respect to the other sections. One skilled in the art will appreciate that any number of sections, including for example, a two-section embodiment to provide a single level rigid plate, may be provided.

Each section of the exemplary two level rigid bone fixation plate 210 may include one or more bores 22 for receiving a bone anchor, such as a bone screw 25, which is effective to mate the bone fixation plate 210 to bone. One or more bores 22 of the exemplary two level rigid bone fixation plate 210 may include a polyaxial bushing 28 to facilitate connection of a bone anchor, e.g., bone screw 25, to the plate 210.

As with the exemplary dynamic bone fixation plates described above, one or more bores 22 of the exemplary two level rigid bone fixation plate 210 may define a bore axis 50 that is varied to provide a range of favored angles for the placement of the bone anchor, e.g., a bone screw 25, into bone.

The exemplary two-level rigid bone fixation plate 210 may include one or more canted sections that are oriented at a cant angle, i.e., an angle other than 0°, to the longitudinal axis of a respective section of the bone fixation plate 210, as described above in connection with the exemplary dynamic bone fixation plates.

Continuing to refer to FIGS. 17 and 18, the exemplary two-level rigid bone fixation plate 210 may include one or more graft windows 220 to facilitate viewing of a graft positioned in the disc space between to adjacent vertebrae to which the bone fixation plate 210 is attached. In particular, a graft window may be positioned between two sections of the bone fixation plate, e.g., between two sets of bores 22 for receiving bone anchors. The size and shape of the graft window 220 may be varied depending on, for example, the size of the plate and the location on the spine at which the bone fixation plate is implanted. One skilled in the art will appreciate that one or more graft windows 220 may be provided on both rigid and dynamic plates of varying size and shape.

While the bone fixation plates of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. A spinal fixation plate comprising:
a first section having at least one bore formed therein for receiving a bone anchor effective to mate the first section to a first vertebra; and
a second section having at least one bore formed therein for receiving a bone anchor effective to mate the second section to a second vertebra, at least one of the first section and the second section having a canted section oriented at a cant angle to at least one other section of the at least one of the first section and the second section, the cant angle being selected to correspond to a geometry of at least one of the first vertebra and the second vertebra.

2. The spinal fixation plate of claim 1, wherein the cant angle is less than approximately 20°.

3. The spinal fixation plate of claim 1, wherein the cant angle is less than approximately 10°.

4. The spinal fixation plate of claim 1, wherein the canted section is at an end of the at least one of the first section and the second section.

5. The spinal fixation plate of claim 4, wherein the canted section is at an end of the first section that is spaced apart along a longitudinal axis of the plate to an end of the second section.

6. The spinal fixation plate of claim 5, wherein the second section includes a second canted section at the end of the second section.

7. The spinal fixation plate of claim 5, wherein the cant angle of the canted section is less than approximately 20° and the cant angle of the second canted section is less than approximately 20°.

8. The spinal fixation plate of claim 5, wherein the cant angle of the canted section is approximately equal to the cant angle of the second canted section.

9. The spinal fixation plate of claim 1, wherein at least one of the second section and the first section is adjustable along a longitudinal axis of the plate with respect to the other section.

10. The spinal fixation plate of claim 9, further comprising a dynamic connection mechanism configured to control relative motion of the second section and the first section, the dynamic connection mechanism comprising a longitudinally oriented slot formed in the first section and a pin fixed to the second section and sized to slidably engage the slot formed in the first section.

11. The spinal fixation plate of claim 1, wherein the second section and the first section are fixed with respect to one another.

12. The spinal fixation plate of claim 1, wherein the at least one bore of the first section and the at least one bore of the second section are positioned at opposing ends of the spinal fixation plate and the at least one bore of the first section has a first bore axis and the at least one bore of the second section has a second bore axis.

13. The spinal fixation plate of claim 12, wherein the first bore axis and the second bore axis intersect at point on a side of the spinal fixation plate distal to the first and second vertebrae.

14. The spinal fixation plate of claim 12, wherein the first bore axis and the second bore axis intersect at point on a side of the spinal fixation plate proximal to the first and second vertebrae.

15. The spinal fixation plate of claim 12, wherein at least one of the first bore axis and the second bore axis is oriented at an angle other than perpendicular to a longitudinal axis of a section of a respective one of the second section and the first section.

16. The spinal fixation plate of claim 15, wherein the angle of the at least one of the first bore axis and the second bore axis is greater than 70° with respect to a longitudinal axis of a section of a respective one of the second section and the first section.

17. The spinal fixation plate of claim 12, wherein the first bore axis and the second bore axis are parallel to one another and oriented at an angle other than perpendicular to a longitudinal axis of the plate.

18. The spinal fixation plate of claim 1, further comprising a polyaxial bushing mounted in at least one bore, the polyaxial bushing configured to permit polyaxial rotation of the bushing within the at least one bore.

19. The spinal fixation plate of claim 1, further comprising at least one opening formed in the plate to permit visualization of a graft positioned between the vertebrae.

20. The spinal fixation plate of claim 1, further comprising at least one intermediate section positioned between the first section and the second section, the at least one intermediate section having at least one bore formed therein for receiving a bone anchor effective to mate the at least one intermediate section to a vertebra between the first vertebra and the second vertebra.

21. The spinal fixation plate of claim 1, further comprising at least one fin projecting from a surface of the plate proximal to a vertebrae to facilitate positioning of the plate relative to a vertebra.

22. A spinal fixation plate having a longitudinal axis, the plate comprising:
a first section having at least one bore formed therein for receiving a bone anchor effective to mate the first section to a first vertebra, the first section having a first canted section oriented at a cant angle to the longitudinal axis of the plate; and
a second section having at least one bore formed therein for receiving a bone anchor effective to mate the second section to a second vertebra, the second section having a second canted section positioned distal to the first canted section along the longitudinal axis of the plate and oriented at the cant angle to the longitudinal axis of the plate, the cant angle being selected to correspond to a geometry of the first and second vertebrae and thereby facilitate mating of the plate to the first and second vertebrae.

23. The spinal fixation plate of claim 22, wherein the cant angle is less than approximately 20°.

24. The spinal fixation plate of claim 22, wherein at least one of the first section and the second section is adjustable along a longitudinal axis of the plate with respect to the other section.

25. The spinal fixation plate of claim 22, further comprising a polyaxial bushing mounted in at least one bore, the polyaxial bushing configured to permit polyaxial rotation of the bushing within the at least one bore.

26. The spinal fixation plate of claim 22, further comprising at least one opening formed in the plate to permit visualization of a graft positioned between the vertebrae.

27. A spinal fixation plate having a longitudinal axis, the spinal fixation plate comprising:
a first section having at least one bore formed therein for receiving a bone anchor effective to mate the first section to a first vertebra;
a second section having at least one bore formed therein for receiving a bone anchor effective to mate the second section to a second vertebra, at least one of the second section and the first section being adjustable with respect to the other section along a longitudinal axis of the plate; and
a polyaxial bushing mounted in at least one bore, the polyaxial bushing configured to permit polyaxial rotation of the bushing within the at least one bore.

28. The spinal fixation plate of claim 27, further comprising a dynamic connection mechanism configured to control relative motion of the second section and the first section, the dynamic connection mechanism comprising a longitudinally oriented slot formed in the first section and a pin fixed to the second section and sized to slidably engage the slot formed in the first section.

29. The spinal fixation plate of claim 27, wherein the at least one bore of the first section and the at least one bore of the second section are positioned at opposing ends of the spinal fixation plate and the at least one bore of the first section has a first bore axis and the at least one bore of the second section has a second bore axis that intersects the first bore axis on a side of the spinal fixation plate distal to the first and second vertebrae.

30. The spinal fixation plate of claim 27, wherein the polyaxial bushing has a slot formed therein to permit radial expansion of the bushing.

31. The spinal fixation plate of claim 27, wherein the polyaxial bushing has a plurality of ridges formed on a radially outer surface of the bushing.

32. The spinal fixation plate of claim 31, wherein the radially outer surface of the bushing is generally spherical in shape.

33. The spinal fixation plate of claim 31, wherein a radially interior surface of the polyaxial bushing defines a passage for receiving a bone anchor, the passage tapering from a distal end of the bushing to a proximal end of the bushing.

34. The spinal fixation plate of claim 31, wherein the polyaxial bushing has a generally smooth radially interior surface that defines a passage for receiving a bone anchor.

35. A spinal fixation plate having a longitudinal axis, the spinal fixation plate comprising:
a first section having at least one bore formed therein for receiving a bone anchor effective to mate the first section to a first vertebra, the at least one bore of the first section having a first bore axis; and
a second section having at least one bore formed therein for receiving a bone anchor effective to mate the second section to a second vertebra, the at least one bore of the second section having a second bore axis that intersects the first bore axis on a side of the spinal fixation plate distal to the first and second vertebra.

36. The spinal fixation plate of claim 35, wherein the at least one bore of the first section is positioned proximate an end on the spinal fixation plate and the at least one bore of the second section is positioned proximate the other end of the spinal fixation plate.

37. The spinal fixation plate of claim 35, wherein at least one of the first bore axis and the second bore axis is oriented at an angle other than perpendicular to the longitudinal axis of the spinal fixation plate.

38. The spinal fixation plate of claim 37, wherein the angle of the at least one of the first bore axis and the second bore axis is greater than 70° with respect to the longitudinal axis of the spinal fixation plate.
